(19) United States
(12) Patent Application Publication
(10) Pub. No.: US 2024/0091473 A1
(43) Pub. Date: Mar. 21, 2024

(54) SYSTEMS AND METHODS OF USING A MECHANICAL VENTILATOR TO FACILITATE THE MOVEMENT OF SECRETIONS

(71) Applicant: Breas Medical AB, Molnlycke (SE)

(72) Inventors: William A. Truschel, Oakmont, PA (US); Michelle Chatwin, Stratford-Upon-Avon (GB)

(21) Appl. No.: 18/055,960

(22) Filed: Nov. 16, 2022

Related U.S. Application Data
(60) Provisional application No. 63/376,012, filed on Sep. 16, 2022.

(51) Int. Cl. A61M 16/00; A61M 16/06

(52) U.S. Cl. CPC A61M 16/024; A61M 16/06; A61M 2016/0033

(57) ABSTRACT

A mucus clearance system and method for automatically adjusting various parameters of a ventilator, including Fall Time, Rise Time, and Inspiratory Time, within a prescribed pressure, which are adjusted and based on sensor input data to achieve a targeted Flow Bias difference or ratio.

### COPYRIGHT INFORMATION

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to facilitating the movement of secretions produced in the throat and lung areas generally in the direction towards a person's throat and mouth areas using a mechanical ventilator.

### BACKGROUND OF THE INVENTION

Secretions in the human airways can impair perfusion, prevent gas humidification via membranes and obstruct normal air flow in breathing. The secretion of mucus can thicken on the walls of the small airways and develop into more serious health problems for sick patients.

Often patients with respiratory insufficiency suffer from respiratory infections, are chronically hyper secretive or have arrived at the condition of respiratory insufficiency through a muscular weakness that also prevents productive coughing to mobilize and eliminate mucus buildup.

These patients who benefit from the assistance of a mechanical ventilator may consequently suffer from worsening mucus buildup because the positive applied pressure at the mouth or tracheal interface tends to push excretions in the caudal (wrong) direction for airway clearance.

It is further realized that the application of mechanical ventilation through an artificial airway often blocks the ability of the patient to extract or swallow elevated airway secretions.

Currently it is common for caregivers to undertake specific, adjunct therapies for mechanically ventilated patients to induce the movement of secretions toward the upper airway such as hyperinflation, PEEP-ZEEP maneuvers, rib cage compression or by employing Mechanical Insufflation Exsufflation (MI-E) devices.

These devices and exercises produce a high expiratory flow rate that shears the mucus away from the cell walls and facilitates movement away from the lungs toward the main bronchus.

However, these techniques, are often invasive and require assistance from trained professionals to implement, which can by a drain on human capital or make homecare solutions difficult.

The present disclosure and embodiments provided below seek to provide a solution that reduces human capital while utilizing current mechanical ventilator equipment to provide a mucus clearing system. These solutions as well as other advantages will become apparent to those skilled in the art from the written description provided.

### SUMMARY OF THE INVENTION

In one embodiment A method of using a ventilator to aid in clearing mucus comprises the steps of: 1) inputting into the ventilator a target flow bias ratio, wherein the target flow bias ratio is in the direction of expiration; 2) measuring a current flow bias ratio of the ventilator, 3) comparing the target flow bias ratio to the measured current flow bias ratio; and 4) modifying at least one of the operational parameters of the ventilator: a) rise time, b) fall time or c) inspiratory time, when the measured current flow bias ratio is not within a predetermined range of the target flow bias ratio.

The method above can further include the step of measuring the fall time of the ventilator and determining if the fall time of the ventilator has reached a pre-determined minimum.

The method above can further include the step of generating a new fall time if the measured fall time has not reached the pre-determined minimum.

The method above can further include the step of generating a new rise time, a new fall time and a new inspiratory time if the measured fall time has reached the pre-determined minimum.

The methods above wherein the ventilator is configured to maintain a prescribed pressure treatment level.

A variation to the method of using the ventilator above, wherein the modification step is configured to incrementally increase the measured flow bias ratio until it achieves the desired target flow bias ratio over a period of breaths. The period of breaths is at least 5 breaths, at least 10 breaths, at least 15 breaths, at least 20 breaths, or at least 25 breaths.

The fall time in the above methods can be reduced so as to increase the measured flow bias ratio. Alternatively, the fall time can be increased so as to reduce the measured flow bias ratio.

In yet another embodiment a mucus clearing assistance system comprised of: a ventilator system configured to provide positive air pressure to a user; one or more sensors configured to detect flow rate associated with the ventilator; and a controller configured to change one or more output parameters associated with the ventilator, wherein the controller has programmable logic or memory and a processing unit configured to perform the following steps: 1) receive a target flow bias ratio input, 2) receive from the one or more sensors, a measured flow rate, 3) determine from the measured flow rate a flow bias ratio, 4) compare the measured flow bias ratio to the target bias ratio, 5) determine if the measured flow bias ratio needs to be increased or decreased, and 6) modifying at least one of: a fall time parameter, a rise time parameter, and an inspiratory time parameter based on the determination to increase or decrease the measured flow bias ratio.

The above mucus clearing assistance system can be designed wherein the fall time and rise time parameters are based on the rate at which each is pressurized or depressurized. The controller can also be configured to modify the pressurization and de-pressurization rates of the ventilator.

The above mucus clearing assistance system can be designed wherein the controller is further configured determine if the fall time of the ventilator has reached a pre-determined minimum.

The above mucus clearing assistance system can be designed wherein the controller can generate a new fall time if the measured fall time has not reached the pre-determined minimum.

The above mucus clearing assistance system can be designed wherein the controller is further configured to perform the step of generating a new rise time, a new fall time and a new inspiratory time if the measured fall time has reached the pre-determined minimum.

These and other embodiments will become apparent to those skilled in the art upon reviewing the rest of the application.

### BRIEF DESCRIPTION OF THE DRAWINGS

The foregoing and other objects, features, and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 illustrates a ventilator system;

FIG. 2 illustrates lungs and the inspiration and expiration flows in and out of the lungs;

FIG. 3 illustrates a schematic of mucus clearing system;

FIGS. 4A-B illustrate various methodologies to set a Target Flow Bias, Measure a current Flow Bias and determine modifications;

FIG. 5 illustrates a schematic that also includes utilizing a Bias Error as part of the determination for updating the ventilator operating parameters;

FIGS. 6A.1-D.5 illustrate various graphs that provide examples of altering ventilator operating parameters in various ways based on the measured Flow Bias and other measure parameters.

### DETAILED DESCRIPTION OF THE INVENTION

Some definitions that will be helpful for this application including the following:

Inspiratory flow refers to the flow of air entering into and flowing towards the lungs. Expiratory flow refers to the flow of air exiting the lungs and flowing towards the glottis.

Rise Time is the rate at which the pressure ramps up to the prescribed or determined pressure level. The pressure generally rises during inspiration. The rise time of pressure can affect the flow rate and in particular the inspiratory flow rate and in more particular the peak inspiratory flow.

Fall Time is the rate at which the pressure ramps down to a determined pressure level. The pressure generally decreases during expiration. The fall time of pressure can affect the flow rate and in particular the expiratory flow rate and in more particular the peak expiratory flow.

Inspiratory Time is the length of time during which the ventilator delivers the inspiratory pressure.

PEEP is positive end-expiratory pressure.

ZEEP is zero end-expiratory pressure.

Hyperinflation of the lungs refers to overfilling of the lungs.

Prescribed Pressure or Pressure Dosage is the amount of pressure that the ventilator ramps up to during use of the ventilator and is usually prescribed by a medical provider. Units of pressure are usually in the form of cm H₂O or centimeters of water column. Prescribed pressures generally range from 5-25 cm H₂O, and generally do not exceed 30 cm H₂O.

Flow Bias refers to the difference between the peak expiratory flow (PEF) and the peak inspiratory flow (PIF) when both flows have been assigned the same directional sign. E.g. If PIF=30 L/min and PEF=−35 L/min then the Flow Bias is 5 L/min.

Flow Bias Ratio refers to the absolute value of the ratio of PEF/PIF.

Target Flow Bias refers to a prescribed difference or ratio of PEF to PIF to encourage airway clearance.

Measured Flow Bias refers to the measured or observed Flow Bias.

Measured Flow Bias Ratio refers to the measured or observed Flow Bias Ratio.

The term significant used throughout this application, whether significantly reduce, significantly improved, significantly worse, or other variation is meant to convey a statistical or mathematical deviation away from a mean or expected mean. This statistical deviation generally means at least one standard deviation or more, whereas, something less than a standard deviation would NOT be considered significant. Using standard deviation is not always robust, therefore, other forms of deviation could be used such as median absolute deviation. In summary, those skilled in the art of statistics would appreciate when a value has a mathematical or statistical variation that is ‘significant.’

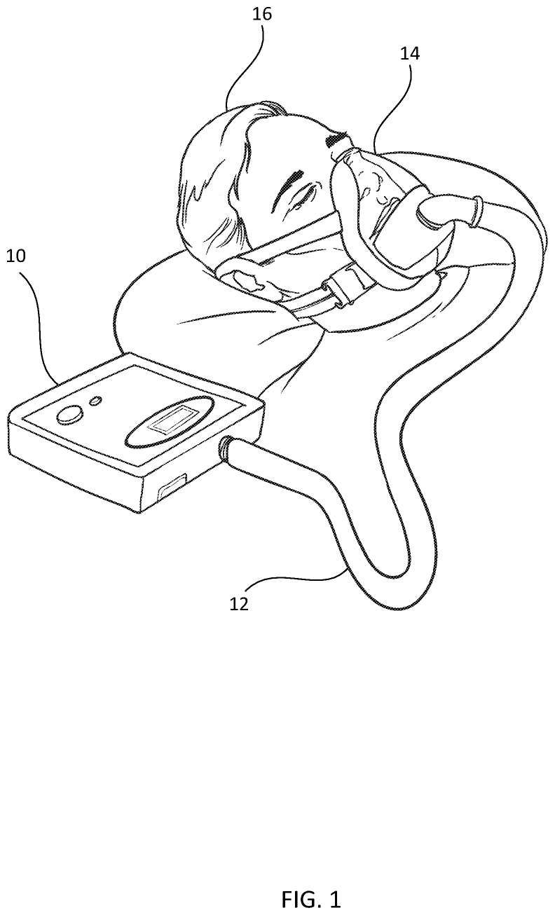

FIG. 1 illustrates a basic ventilator system 10 that provides pressurized air through the tube 12 into an airway adaptor 14, such as a mask, to the user/patient 16. In some instances, a mask is not used, where the tube is directly fed into the trachea, such as a tracheostomy.

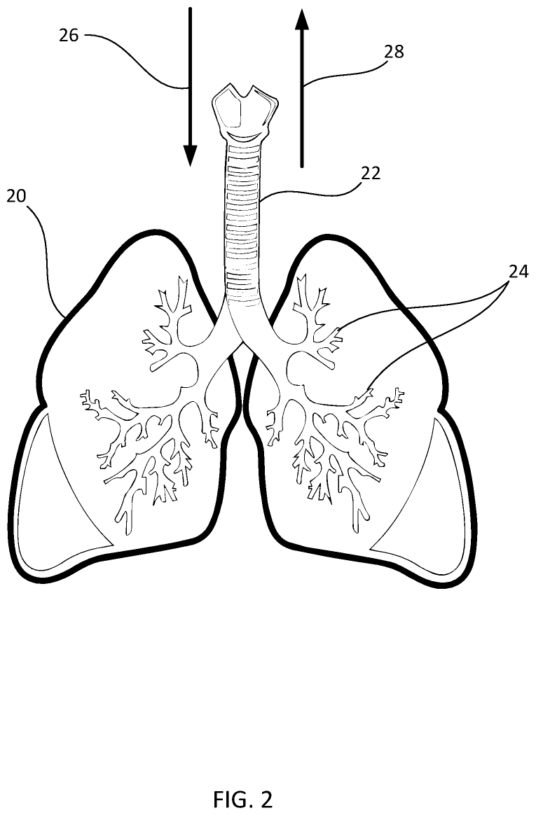

FIG. 2 illustrates lungs 20, including the trachea 22 and the bronchi of the lungs 24. The inspiration flow path 26 travels into the trachea 22 and into bronchi 24, whereas the expiration flow path 28 travels or flows out from the lungs 20 and bronchi 24 into and out of the trachea 22.

It should be understood that the volume of air inhaled or inspired by a user/patient, generally, and should be equal the volume of air exhaled or expired by a user/patient. If not, the user/patient will have problems. Thus, when user/patients are on ventilator machines, they are designed to maintain an equal volume of air going in and coming out of the lungs.

It is well known that in second order pneumatic systems (a second order pneumatic system is a system in which the dynamics within the system are determined by the pressure, flow and the first derivative of pressure and flow only), the peak flow is determined by the rate of change in the driving pressure. The rate of change in driving pressure is programmed in mechanical ventilator settings by the “Rise Time” and the “Fall Rate” settings. The “Rise Time” affects the peak inspiratory flow because it determines the rate of pressurization at the start of inspiration. The “Fall Time” affects the peak expiratory flow because it determines the rate of depressurization during the start of expiration.

Using an analogy of a leaf blower, the greater flow rate from a more powerful leaf blower is more likely to lift fallen leaves from the ground to gather them for disposal. In this invention, the higher peak expiratory flows induced by fast depressurization of the airways is more likely to lift secretions from the airway walls to gather them near the glottis where they can be removed.

In mechanical ventilation therapy, it is often the case that during assisted inspiration, the leaf blower is pointed in the wrong direction when air is forced into the lungs and it becomes harder for the patient to manage the upward movement of secretions through natural means or through coughing. Thus, from this analogy, it is understood that by controlling or changing the “Rise Time” and “Fall Time” the overall affect of pointing the leaf blower in the correct direction can occur, which is determined by the Target Bias Flow Ratio or Difference.

As noted in the background, previous manual techniques, such as expiratory rib cage compression (ERCC) or monitored techniques, such as hyperinflation, which sometimes include temporarily setting the peak inspiratory pressure to 40 cm H₂O, are only designed to be used under monitored situations. This is because each of these techniques require additional tools that medical professionals need to implement, and/or require medical professional monitoring, because the techniques are extreme in nature and are not meant to be utilized constantly. In contrast, the methods and systems discussed herein help to eliminate some of the additional medical professional monitoring and/or implementation, because they can be implemented within the prescribed pressure limits a user/patient is meant to be on over longer periods of time. For example, hyperinflation, which uses pressures of 40 cm H₂O, is well outside the normal prescribed range of 5-25 cm H₂O for consistent ventilator use. Thus, by automatically modifying the Rise Time, Fall Time and Inspiratory Time parameters, without changing the prescribed pressure the methods and systems described herein can achieve the desired Flow Bias difference or ratio over longer periods of time and avoid additional medical professional time or techniques implemented by medical professionals. In some cases, the consistency of the methods and systems described herein become more effective for clearing mucus from a user/patient, less costly, require less medical professional labor intensive, and can be adapted to utilize existing ventilators in the market.

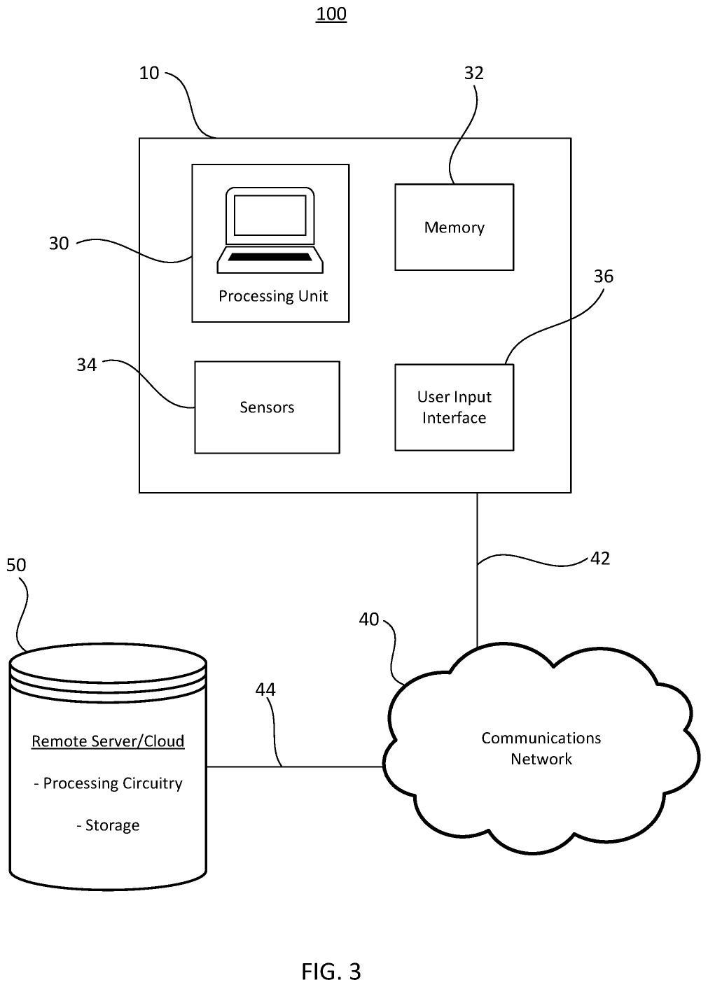

FIG. 3 illustrates a schematic of mucus clearing system 100 that includes a ventilator system 10, which includes a processing unit 30 configured to receive input parameters, such as target Flow Bias parameters via a user/patient input interface 36, implement algorithms, protocols, as well as direct and analyze sensor data captured by sensors 34, recall and place data into memory 32, and direct communications over a network 40 to remote server/cloud 50 that also includes processing circuitry and storage. Directed communications 42 can be made to and from the communications network, which can make directed communications 44 to and from the remote server/cloud 50. Cloud computing is generally understood in the art to mean the delivery of computing services—including servers, storage, databases, networking, software, analytics, and intelligence—over the Internet (“the cloud”) to offer faster innovation, flexible resources, and economies of scale.

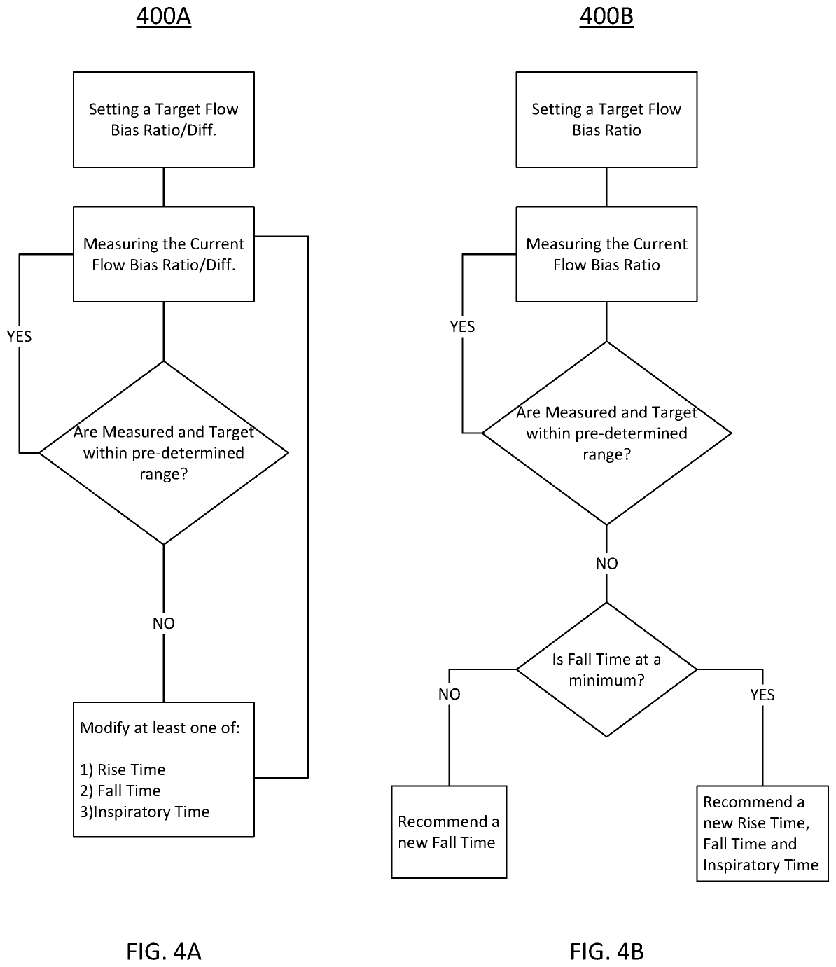

FIGS. 4A-B illustrate various methodologies set a target Flow Bias, Measure a current Flow Bias and determine (and make) modifications needed until the measured Flow Bias (ratio or difference) are within a pre-determined range of the Target Flow Bias (ratio or difference).

Referring to flow chart 400A in FIG. 4A, a user, such as a medical professional, sets a Target Flow Bias or Difference for the operation of the ventilator to achieve. One example target range of the Flow Bias ratio could be 1.1. Other target flow bias ratios could be 1.05, 1.06, 1.07, 1.08, 1.09, 1.11, 1.12, 1.13, 1.14 and 1.15. The target flow difference could be at least 17 L/min, and alternative differences could be at least 16 L/min, at least 18 L/min, and at least 19 L/min. Once the Target Flow Bias is entered, the next step is to begin measuring flow to determine the current Flow Bias. This can be done using one or more of the sensors associated with the ventilator. Flow and pressure are common measurements most ventilators are configured to measure. Measuring pressure is often used to estimate leak in a given ventilator system, which provides a more accurate estimate of the actual flow being measured.

These above measurements can be used to determine the PEF and PIF, which can then be used to determine the ratio of PEF/PIF or difference of PEF-PIF. Once the measured Flow Bias is determined, then the next step is to determine if it is within a pre-determined range of the Target Flow Bias. This pre-determined range could be an actual number or it could be determined whether it is statistically insignificant or not based on the Target Flow Bias number entered. For example, if the Target Flow Bias ratio is 1.1 and the measured Flow Bias ratio is 1.0998, this is likely to be within the pre-determined range and thus would require no modification of the current operating parameters of the ventilator as shown through the decision step. Thus, continued measuring would occur until the difference between Target and Measured Flow Bias is outside of the pre-determined range.

When it is determined that the Measured is outside of the pre-determined range, depending on whether that ratio or difference is higher or lower than the target then the next step would be to modify at least one of the following parameters: 1) Rise Time, 2) Fall Time, or 3) Inspiratory Time. In some instances, it may be necessary to adjust more than one of the above parameters. Once the parameter(s) are adjusted the next step is to allow one or more cycles to pass and begin measuring the Flow Bias with the updated parameters. These steps are repeated until the optimal parameters are achieved. It should be noted that a user's/patient's breathing can change over the course of a day or night, as different stages of sleep or awakeness can alter the breathing patterns. Thus, the parameters can constantly and automatically be updating throughout the day based on the user's/patient's biocycle.

Referring to flow chart 400B in FIG. 4B, a user, such as a medical professional, again sets a Target Flow Bias Ratio or Target Bias Flow Difference for the operation of the ventilator to achieve. The first couple steps are similar to that of flowchart 400A; however, once it is determined that the Measured to Target Flow Bias is outside of the pre-determined range, an intermediary step of determining if the current Fall Time is at a minimum threshold or not. If it is not, then the method recommends updating a new Rise Time. If it is at the minimum threshold then the method can recommend a new Rise Time, Fall Time and Inspiratory Time. After each of the recommendations, the method can include implementing those new recommendations and begin a new cycle of measuring the Flow Bias and comparing it to the Target Flow Bias.

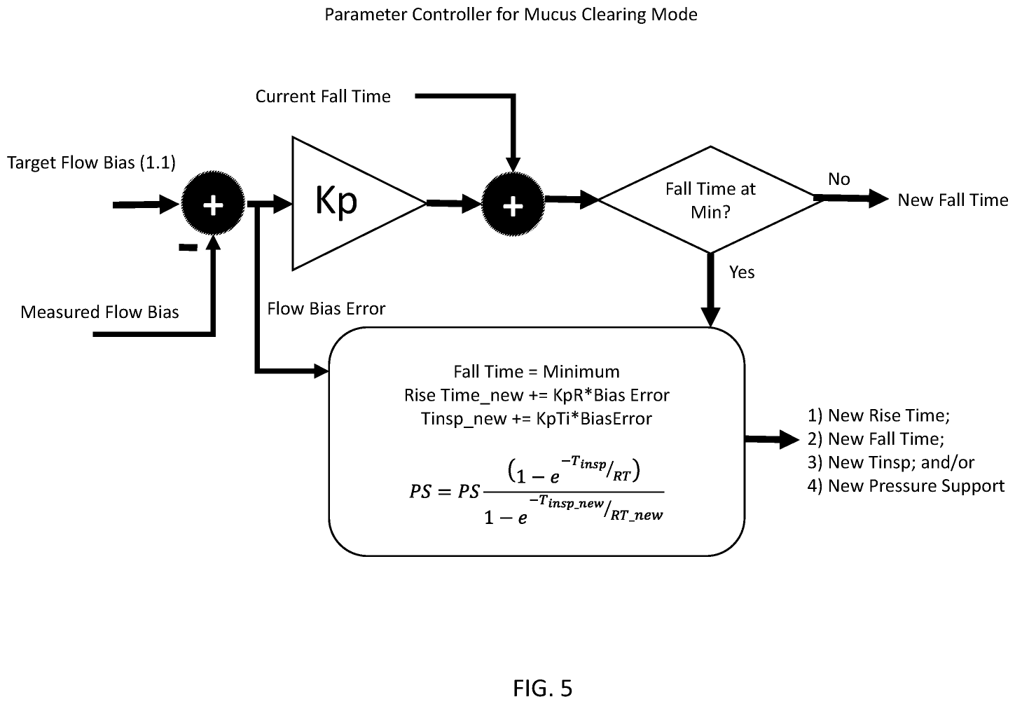

FIG. 5 illustrates a schematic that also includes utilizing a Bias Error as part of the determination for updating the ventilator operating parameters. Here the Target Flow Bias is set at the ratio of 1.1 and compared with the Measured Flow Bias Ratio. Kp is indicative of the proportional gain controller, which helps to modify the speed or rate at which the ventilator pressurizes and de-pressurizes. As noted above, the rate at which the ventilator pressurizes effects the speed of the flow rate during the Fall Time or Rise Time, which ultimately determines the PEF and PIF which are used to calculate the Flow Bias ratio. If the Flow Bias Ratio is optimized appropriately it can help the user/patient clear mucus from there lung, trachea and other areas without the need to have, or at least reduce the number of, additional therapies to manage mucus buildup in a user/patient. A Bias Error can be determined using some of the equations shown. Similar to Flowchart 400B, the current Fall Time can be measured and used to determine if it is at minimum threshold or not. If not, then a new Fall Time is recommended and if so, then it can be used to initiate the Bias Error equations to determine a new Rise Time, new Fall Time, New Inspiratory Time and/or even new Pressure Support.

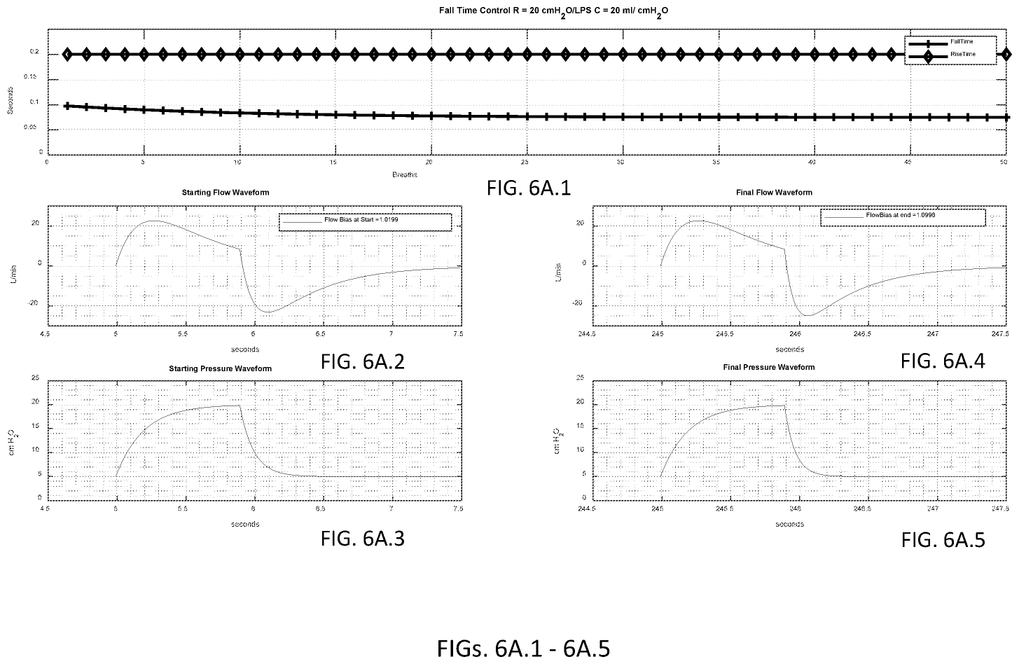

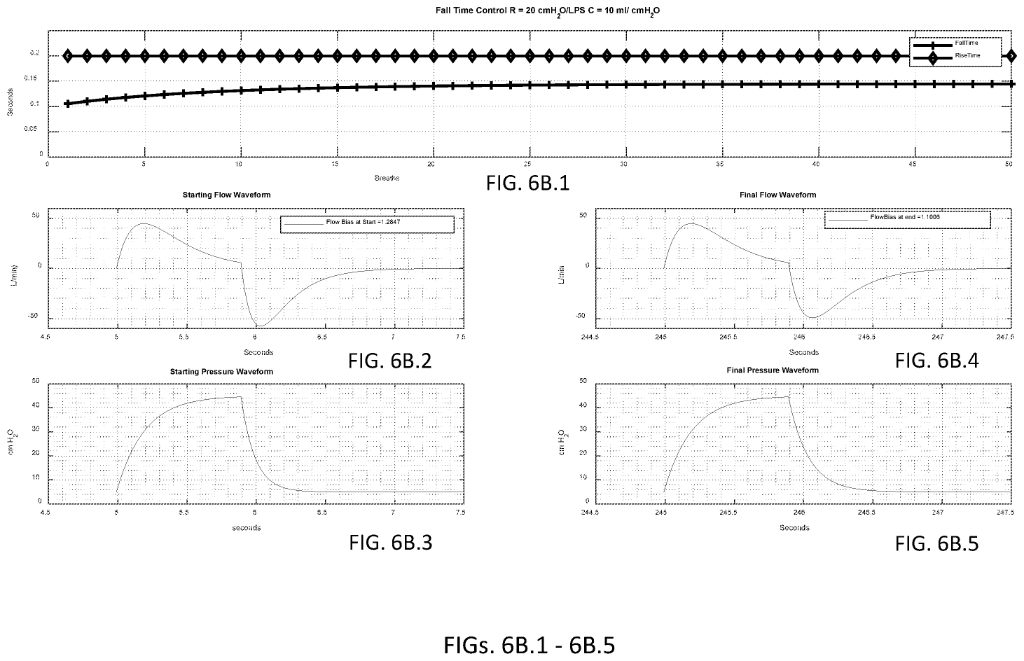

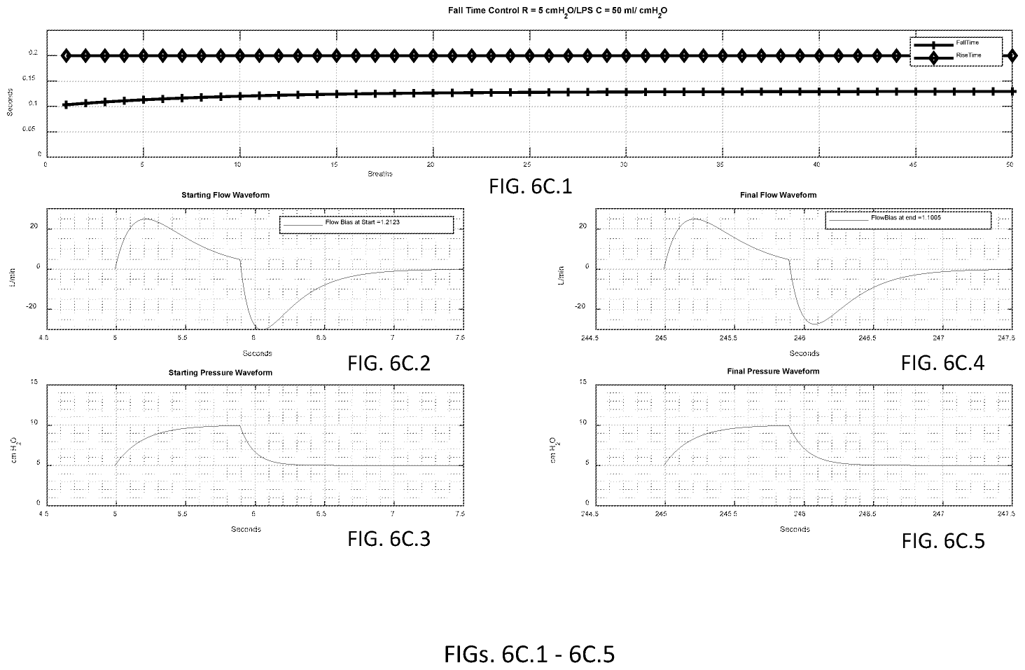

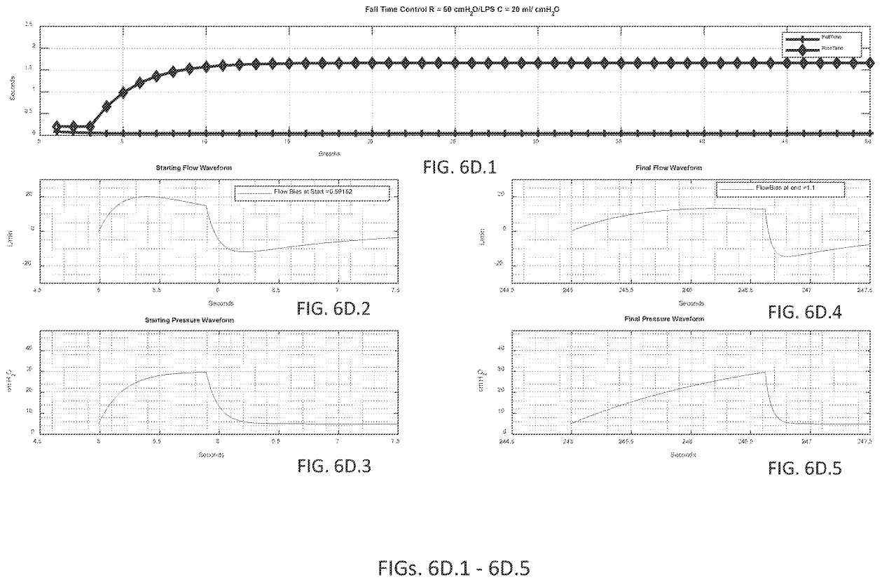

To further illustrate the effects of the above system and methods FIGS. 6A.1-D.5 have been provided to illustrate various graphs that provide examples of altering ventilator operating parameters in various ways based on the measured Flow Bias and other measure parameters.

For example, in FIGS. 6A.1-A.5, here the initial Flow Bias ratio is measured at 1.0199 and the desired Target Flow Bias ratio is 1.1. As a result, the controller reduces the Fall Time operational parameter of the ventilator, to bring the measured Flow Bias ratio up. In FIG. 6A.1, it can be readily shown where the Fall Time decreases from an initial 0.1 seconds to 0.07 seconds after about 10 breaths in the cycle occur. The effect that this change has is shown by comparing the measured Starting Flow Waveform (FIG. 6A.2) and Starting Pressure Waveform (FIG. 6A.3) graphs on the left side with the Final Flow Waveform (FIG. 6A.4) and Final Pressure Waveform (FIG. 6A.5) graphs on the right. The resulting end ratio or new measured ratio is 1.0996 as shown FIG. 6A.4.

In another example, in FIGS. 6B.1-B.5, here the initial measured Flow Bias ratio is 1.2847. The desired Target Flow Bias ratio is 1.1, so the controller increases the Fall Time, to bring the initial measured Flow Bias ratio down. In FIG. 6B.1, it can be readily shown where the Fall Time increase from an initial 0.1 seconds to 0.13 seconds after about 10 breaths and up to 0.14 seconds after about 25 breaths occur in the cycle. The effect that this change has is shown again by comparing the measured Starting Flow Waveform (FIG. 6B.2) and Starting Pressure Waveform (FIG. 6B.3) graphs on the left side with the Final Flow Waveform (FIG. 6B.4) and Final Pressure Waveform (FIG. 6B.5) graphs on the right. The resulting end ratio or new measured ratio is 1.1006 as shown FIG. 6B.4.

In yet another example, in FIGS. 6C.1-C.5, here the initial measured Flow Bias is around 1.2. The desired Target Flow Bias ratio is 1.1, so the controller increases the Fall Time, to bring the initial measured Flow Bias ratio down. In FIG. 6C.1, it can be readily shown where the Fall Time increase from an initial 0.1 seconds to around 0.12 seconds after about 10 breaths and up to 0.13 after about 25 breaths occur in the cycle. The effect that this change has is shown again by comparing the measured Starting Flow Waveform (FIG. 6C.2) and Starting Pressure Waveform (FIG. 6C.3) graphs on the left side with the Final Flow Waveform (FIG. 6C.4) and Final Pressure Waveform (FIG. 6C.5) graphs on the right. The resulting end ratio or new measured ratio is around 1.1, as shown FIG. 6C.4.

In yet one more example, in FIGS. 6D.1-D.5, here the initial measured Flow Bias ratio is around 0.6. The desired Target Flow Bias ratio is 1.1, so the controller causes the Fall Time to go to the minimum value and increases the Rise Time, to bring the measured Flow Bias ratio up. In FIG. 6D.1, it can be readily shown where the Fall Time decreases from an initial 0.1 seconds to the predetermined minimum fall time after about 10 breaths and as according the algorithm in FIG. 5, the rise time, pressure support (PS), and inspiratory time (Tinsp) are subsequently adjusted. The Rise Time increases from 0.2 seconds to 1.6 seconds between 10 to 15 breaths in the cycle. The pressure support and inspiratory time increase to maintain a consistent peak pressure according to the relationship in FIG. 5. The effect that this change has is shown again by comparing the measured Starting Flow Waveform (FIG. 6D.2) and Starting Pressure Waveform (FIG. 6D.3) graphs on the left side with the Final Flow Waveform (FIG. 6D.4) and Final Pressure Waveform (FIG. 6D.5) graphs on the right. The resulting end ratio or new measured ratio is 1.1 as shown FIG. 6D.4. Here the effective pressure support also remains the same, while the inspiratory time has increased. This can be seen because the maximum pressure delivered is the same for the starting and final waveform graphs.

As noted above, by focusing on where the Measured Flow Bias is initially with respect to the Target Flow Bias, the methods and systems described herein can adjust one or more ventilator operating parameters within the prescribed pressure, to achieve operating near the Targeted Flow Bias ratio or difference. When the Flow Bias ratio or difference is set appropriately, this can help move mucus out of the lungs and trachea regions. These methods and systems can be implemented to automatically adjust the operating parameters of the ventilator, when necessary, thus allowing a constant and consistent Flow Bias ratio to be applied to the user, which consistently moves mucus out of the lungs and trachea regions.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

What is claimed is:

1. A method of using a ventilator to aid in clearing mucus comprising the steps of:
inputting into the ventilator a target flow bias ratio, wherein the target flow bias ratio is in the direction of expiration;
measuring a current flow bias ratio of the ventilator,
comparing the target flow bias ratio to the measured current flow bias ratio; and
modifying at least one of the operational parameters of the ventilator: rise time, fall time, and inspiratory time when the measured current flow bias ratio is not within a predetermined range of the target flow bias ratio.

2. The method of using a ventilator to aid in clearing mucus of claim 1, further including the step of measuring the fall time of the ventilator and determining if the fall time of the ventilator has reached a pre-determined minimum.

3. The method of using a ventilator to aid in clearing mucus of claim 2, further comprising the step of generating a new fall time if the measured fall time has not reached the pre-determined minimum.

4. The method of using a ventilator to aid in clearing mucus of claim 2, further comprising the step of generating a new rise time, a new fall time and a new inspiratory time if the measured fall time has reached the pre-determined minimum.

5. The method of using a ventilator to aid in clearing mucus of claim 3, wherein the ventilator is configured to maintain a prescribed pressure treatment level.

6. The method of using a ventilator to aid in clearing mucus of claim 1, wherein the ventilator is configured to maintain a prescribed pressure treatment level.

7. The method of using a ventilator to aid in clearing mucus of claim 1, wherein the modification step is configured to incrementally increase the measured flow bias ratio until it achieves the desired target flow bias ratio over a period of breaths.

8. The method of using a ventilator to aid in clearing mucus of claim 7, wherein the period of breaths is at least 5 breaths, at least 10 breaths, at least 15 breaths, at least 20 breaths, or at least 25 breaths.

9. The method of using a ventilator to aid in clearing mucus of claim 1, wherein the fall time is reduced so as to increase the measured flow bias ratio.

10. The method of using a ventilator to aid in clearing mucus of claim 1, wherein the fall time is increased so as to reduce the measured flow bias ratio.

11. A mucus clearing assistance system comprised of:
a ventilator system configured to provide positive air pressure to a user;
one or more sensors configured to detect flow rate associated with the ventilator; and
a controller configured to change one or more output parameters associated with the ventilator, wherein the controller has programmable logic or memory and a processing unit configured to perform the following steps:
receive a target flow bias ratio input,
receive from the one or more sensors, a measured flow rate,
determine from the measured flow rate a flow bias ratio,
compare the measured flow bias ratio to the target bias ratio, determine if the measured flow bias ratio needs to be increased or decreased, and
modifying at least one of: a fall time parameter, a rise time parameter, and an inspiratory time parameter based on the determination to increase or decrease the measured flow bias ratio.

12. The mucus clearing assistance system of claim 11, wherein the fall time and rise time parameters are based on the rate at which each is pressurized or depressurized.

13. The mucus clearing assistance system of claim 12, wherein the controller is configured to modify the pressurization and de-pressurization rates of the ventilator.

14. The mucus clearing assistance system of claim 11, wherein the controller is further configured determine if the fall time of the ventilator has reached a pre-determined minimum.

15. The mucus clearing assistance system of claim 14, wherein the controller can generate a new fall time if the measured fall time has not reached the pre-determined minimum.

16. The mucus clearing assistance system of claim 14, wherein the controller is further configured to perform the step of generating a new rise time, a new fall time and a new inspiratory time if the measured fall time has reached the pre-determined minimum.
